(19) United States
(12) Patent Application Publication
(10) Pub. No.: US 2024/0287531 A1
(43) Pub. Date: Aug. 29, 2024

(54) RECOMBINANT MICROORGANISM FOR PRODUCING CARNOSINE, HISTIDINE AND BETA-ALANINE AND METHOD FOR PRODUCING CARNOSINE, HISTIDINE AND BETA-ALANINE BY USING SAME

(71) Applicant: KOREA UNIVERSITY RESEARCH AND BUSINESS FOUNDATION, Seoul (KR)

(72) Inventors: Sung Ok HAN, Seoul (KR); Minhye KIM, Anyang-si (KR); Young Jin KO, Chuncheon-si (KR)

(73) Assignee: KOREA UNIVERSITY RESEARCH AND BUSINESS FOUNDATION, Seoul (KR)

(21) Appl. No.: 18/290,193

(22) PCT Filed: May 6, 2022
(86) PCT No.: PCT/KR2022/006476

§ 371 Date: Nov. 10, 2023

(30) Foreign Application Priority Data
May 11, 2021 (KR) 10-2021-0060669

(51) Int. Cl. C12N 15/77; C12N 9/00; C12N 9/10; C12N 9/12; C12N 9/88; C12N 9/90; C12P 13/06; C12P 13/24; C12P 17/10

(52) U.S. Cl. CPC C12N 15/77; C12N 9/1077; C12N 9/1235; C12N 9/88; C12N 9/90; C12N 9/93; C12P 13/06; C12P 13/24; C12P 17/10; C12Y 204/02017; C12Y 207/06005; C12Y 401/01011; C12Y 503/01009; C12Y 603/02011

(57) ABSTRACT

Provided is a recombinant microorganism for producing carnosine, histidine and beta-alanine and a method for producing carnosine, histidine and beta-alanine by using same and, more particularly, to: a recombinant microorganism for high production of carnosine, histidine and beta-alanine produced through the redesign of metabolic pathways; a method for producing same; and a method for producing carnosine, histidine and beta-alanine by using same. According to the present invention, in a microorganism capable of producing histidine and beta-alanine, by enhancing the pentose phosphate pathways through the replacement of a pentose phosphate pathway-related operon gene with a highly expressing synthetic promoter and the replacement of a pgi gene with an initiation codon, and inducing enhancement of the production of histidine and beta-alanine through the overexpression of genes on histidine and beta-alanine metabolic pathways, respectively, it is possible to develop a recombinant microorganism for high production of histidine and beta-alanine.

### TECHNICAL FIELD

The present invention relates to a recombinant microorganism for producing carnosine, histidine and beta-alanine and a method for producing carnosine, histidine and beta-alanine by using same and, more particularly, to: a recombinant microorganism for high production of carnosine, histidine and beta-alanine produced through the redesign of metabolic pathways; a method for producing same; and a method for producing carnosine, histidine and beta-alanine by using same.

### BACKGROUND ART

L-histidine is one of 20 protein amino acids, and an essential amino acid, which is not synthesized through assimilation in the human body, so that animals such as humans necessarily take proteins containing the histidine. Therefore, the biosynthesis of histidine has been widely studied in prokaryotes, including E. Coli, in which the histidine biosynthesis occurs through a 10-step process using protein (enzyme) products of 8 genes to finally synthesize histidine using phosphoribosyl pyrophosphate (PRPP) as a starting material. The biosynthesis of histidine consumes energy because the ATP-phosphoribosyl transferase that initiates the action requires ATP. Additionally, since the transferase is a rate-determining enzyme, the transferase is inhibited by feedback and its activity is inhibited in the presence of histidine.

The histidine is also converted into other amines with biological activity, and is specifically a precursor to histamine, which plays an important role in inflammatory reactions as a type of immune stimulant. A histidine ammonia-cleavage enzyme decomposes histidine into urocanic acid and ammonia, and defects in the enzyme are known to cause histidinemia, which is a very rare metabolic disorder. In addition, the histidine may be converted into 3-methyl histidine, which is a biomarker for skeletal muscle damage and acts with specific methyl transferase. Furthermore, the histidine is known to function as a precursor in the biosynthesis of carnosine, which is a dipeptide found in skeletal muscle.

Beta-alanine (β-alanine) is a naturally occurring β-amino acid with an amino group bound to the β carbon, and is called 3-aminopropanoic acid in IUPAC nomenclature. The beta-alanine is produced by the decomposition of dihydrouracil and carnosine, and β-alanine ethyl ester is hydrolyzed in the body to produce beta-alanine. In addition, industrially, the beta-alanine is produced through the reaction of ammonia and β-propiolactone.

The beta-alanine is not found in major proteins or enzymes, but is a component of naturally occurring peptides, carnosine and anserine. In addition, the beta-alanine is a component of pantothenic acid (vitamin B5), which is a component of coenzyme A, and is metabolized into acetic acid under normal conditions. The beta-alanine is a rate-limiting precursor of carnosine, in which the level of carnosine is limited by the amount of available beta-alanine other than histidine. Accordingly, beta-alanine supplements were found to increase the concentration of carnosine in muscles, reduce the fatigue in athletes, and increase overall muscle activity. In addition, it has been reported that simply supplementing with only carnosine is not more effective than supplementing with only beta-alanine because carnosine is decomposed into histidine and beta-alanine during the digestive process when taken orally. Therefore, when taking carnosine, only about 40% of the dose based on the weight may be used as beta-alanine.

Carnosine, which is a dipeptide produced by the condensation reaction of histidine and beta-alanine, is found in large quantities in muscles and brain, and through various studies, the antioxidant, anti-radical and anti-inflammatory activities thereof have been reported. In addition, the carnosine acts as an anti-glycation agent to reduce the rate of formation of advanced glycation end-products, such as materials that may be a factor in the development or worsening of many degenerative diseases such as diabetes, atherosclerosis, chronic renal failure and Alzheimer's disease. In addition, in several preclinical studies, its neuroprotective effect has been proven and its effectiveness in improving physical performance, etc. have been known, and thus, recently, in various fields such as the food, medicine, and feed industries, its need has increased and the resulting demand has also increased.

Accordingly, in the related art, in order to industrially produce carnosine, methods for producing the carnosine chemically from phthalic anhydride and hydrazine, or enzymatically using β-aminopeptidase have been used. However, among the methods, in the case of food- and enzyme-based synthesis methods, there is a problem of containing impurities such as antibiotics, hormones, and amino acids due to the use of animal-derived raw materials, and in the case of chemical methods, there is a problem in environmental pollution due to chemical waste and organic solvents. Therefore, there is an urgent need to develop an eco-friendly carnosine synthesis technology capable of replacing the conventional carnosine synthesis methods described above.

### DISCLOSURE

### Technical Problem

Accordingly, the present inventors developed novel recombinant strains capable of producing high levels of carnosine, histidine, and beta-alanine, respectively, by redesigning related metabolic pathways for the production of carnosine using metabolic engineering technology from Corynebacterium glutamicum, which was a generally recognized as safe (GRAS) strain certified by the FDA, in order to develop an eco-friendly carnosine production technology capable of overcoming the limitations in the related art, and then completed the present invention.

An object of the present invention is to provide a recombinant microorganism for high production of carnosine.

Another object of the present invention is to provide a recombinant microorganism for high production of histidine.

Yet another object of the present invention is to provide a recombinant microorganism for high production of beta-alanine.

Another object of the present invention is to provide a method for producing the recombinant microorganism for high production of carnosine.

Yet another object of the present invention is to provide a method for producing carnosine including culturing the recombinant microorganism for high production of carnosine.

Yet another object of the present invention is to provide a method for producing histidine including culturing the recombinant microorganism for high production of histidine.

Yet another object of the present invention is to provide a method for producing beta-alanine including culturing the recombinant microorganism for high production of beta-alanine.

However, technical objects of the present invention are not limited to the aforementioned purpose and other objects which are not mentioned may be clearly understood to those skilled in the art from the following description.

### Technical Solution

One aspect of the present invention provides a recombinant microorganism for high production of carnosine, in which a pentose phosphate pathway is enhanced; and a mammal-derived carnosine synthase 1 (Carns1) gene is introduced.

In one embodiment of the present invention, the recombinant microorganism may be additionally enhanced with one or more of an L-histidine biosynthetic pathway and a beta-alanine biosynthetic pathway.

In another embodiment of the present invention, the Carns1 gene may consist of a nucleotide sequence represented by SEQ ID NO: 22.

Another aspect of the present invention provides a recombinant microorganism for high production of histidine, in which one or more of pentose phosphate pathways and an L-histidine biosynthetic pathway are enhanced.

Another aspect of the present invention provides a recombinant microorganism for high production of beta-alanine, in which one or more of pentose phosphate pathways and a beta-alanine biosynthetic pathway are enhanced.

In one embodiment of the present invention, the pentose phosphate pathways may be enhanced by the replacement of a promoter of an operon-type gene with a highly expressing synthetic promoter, the change of a glucose-6-phosphate isomerase (pgi) gene into an initiation codon, or a combination thereof.

In another embodiment of the present invention, the highly expressing synthetic promoter may be H36.

In yet another embodiment of the present invention, the initiation codon of the pgi gene may be changed from ATG to GTG.

In yet another embodiment of the present invention, the histidine biosynthetic pathway may be enhanced by the overexpression of an ATP phosphoribosyltransferase (HisG) gene, the overexpression of a GTP pyrophosphokinase (Rel) gene, or a combination thereof.

In yet another embodiment of the present invention, the HisG and Rel genes may consist of nucleotide sequences represented by SEQ ID NOs: 13 and 14, respectively.

In yet another embodiment of the present invention, the beta-alanine biosynthetic pathway may be enhanced by the overexpression of an aspartate 1-decarboxylase (PanD) gene.

In yet another embodiment of the present invention, the PanD gene may consist of a nucleotide sequence represented by SEQ ID NO: 19.

In yet another embodiment of the present invention, the recombinant microorganism may be derived from Corynebacterium glutamicum.

Another aspect of the present invention provides a method for producing the recombinant microorganism for high production of carnosine including the following steps:
(a) enhancing pentose phosphate pathways through the replacement of a promoter of an operon-type gene with a highly expressing synthetic promoter, the change of a glucose-6-phosphate isomerase (pgi) gene into an initiation codon, or a combination thereof in microorganisms having glutamic acid productivity; and
(b) introducing a mammal-derived carnosine synthase 1 (Carns 1) gene.

In one embodiment of the present invention, the production method may further include the overexpression of ATP phosphoribosyltransferase (HisG) and GTP pyrophosphokinase (Rel) genes, the overexpression of the aspartate 1-decarboxylase (PanD) gene, or a combination thereof.

In another embodiment of the present invention, the microorganism having the glutamic acid productivity may be Corynebacterium glutamicum.

In yet another embodiment of the present invention, in step (a), the highly expressing synthetic promoter may be H36.

In yet another embodiment of the present invention, in step (a), the initiation codon of the pgi gene may be changed from ATG to GTG.

In yet another embodiment of the present invention, the mammal-derived Carns1 gene may be derived from a mouse (Mus musculus).

Yet another aspect of the present invention provides a method for producing carnosine including culturing the recombinant microorganism for high production of carnosine.

In one embodiment of the present invention, the culture may be performed through fed-batch fermentation.

Yet another aspect of the present invention provides a method for producing histidine (L-Histidine) including culturing the recombinant microorganism for high production of histidine.

Yet another aspect of the present invention provides a method for producing beta-alanine including culturing the recombinant microorganism for high production of beta-alanine.

### Advantageous Effects

According to the present invention, in a microorganism capable of producing histidine and beta-alanine, by enhancing the pentose phosphate pathways through the replacement of a pentose phosphate pathway-related operon gene with a highly expressing synthetic promoter and the replacement of a pgi gene with an initiation codon, and inducing enhancement of the production of histidine and beta-alanine through the overexpression of genes on histidine and beta-alanine metabolic pathways, respectively, it is possible to develop a recombinant microorganism for high production of histidine and beta-alanine. In addition, by introducing Carns1, which is a mammal-derived carnosine synthase gene, carnosine can be mass-produced with a high yield in an eco-friendly manner while overcoming the limitations of conventional methods for synthesizing carnosine.

### DESCRIPTION OF DRAWINGS

FIG. 1 illustrates biosynthetic pathways for sustainable production of carnosine in a recombinant microorganism according to the present invention, in which a red pathway represents an enhanced histidine biosynthetic pathway, green represents an enhanced pentose phosphate pathway, blue represents an enhanced beta-alanine biosynthetic pathway, and purple represents a newly introduced mammal-derived carnosine synthetic pathway.

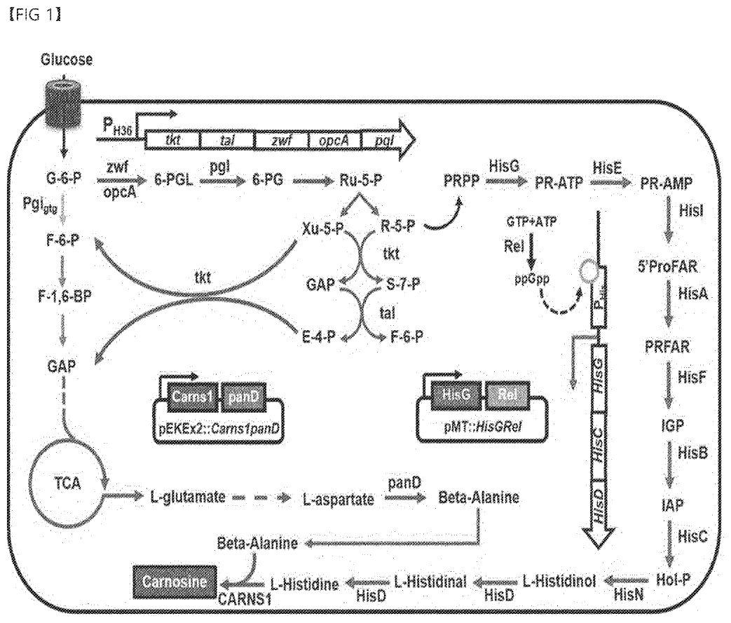

FIG. 2 illustrates a structure of a pK19mobsacB LA-H36-RA vector constructed to replace a tkt promoter with a synthetic promoter H36 in Corynebacterium glutamicum genomic DNA.

FIG. 3 illustrates a structure of a pJYS2::crRNA-pgi vector constructed to replace an initiation codon of a pgi gene with gtg in Corynebacterium glutamicum genomic DNA.

FIG. 4 illustrates structures of pMT-tac::HisG, pMT-tac::Rel, pMT-tac::HisGRel, pEKEx2::Carns1 and pEKEx2::Carns1panD recombinant vectors constructed for enhancing a histidine biosynthetic pathway, enhancing a beta-alanine biosynthetic pathway, and/or inducing a carnosine synthetic pathway in Corynebacterium glutamicum, respectively.

FIG. 5 illustrates results of measuring productions of histidine and carnosine in a control Corynebacterium glutamicum (Car0), a recombinant strain (Car1) in which a carnosine synthetic pathway is introduced by introducing a Carns1 gene into a control strain, a strain (Car2) in which a pentose phosphate pathway is enhanced by replacing a tkt promoter of Corynebacterium glutamicum with a synthetic promoter H36, and a recombinant strain (Car3) in which a pentose phosphate pathway is enhanced and a carnosine synthetic pathway is introduced, respectively.

FIG. 6 illustrates growth curves and histidine productivities measured for culture periods of recombinant strains (Car5, Car6, and Car7, respectively) in which a histidine biosynthetic pathway is enhanced by overexpressing HisG, Rel, or HisG and Rel in control Corynebacterium glutamicum (Car4), and recombinant strains (Car9, Car10, and Car11, respectively) with HisG, Rel, or HisG and Rel overexpressed in a recombinant strain (Car8) in which a pentose phosphate pathway is enhanced by replacing a tkt promoter of Corynebacterium glutamicum with a highly expressing synthetic promoter H36.

FIG. 7 illustrates results of measuring productions of histidine, beta-alanine, and carnosine in each strain after culturing a recombinant strain (Car12) in which a pentose phosphate pathway is enhanced and a carnosine synthetic pathway is introduced by introducing a Carns1 gene; a recombinant strain (Car13) in which a pentose phosphate pathway and a beta-alanine biosynthetic pathway are enhanced and a carnosine synthetic pathway is introduced; a recombinant strain (Car14) in which a pentose phosphate pathway and a histidine biosynthetic pathway are enhanced and a carnosine synthetic pathway is introduced; and a recombinant strain (Car15) in which a pentose phosphate pathway and beta-alanine and histidine biosynthetic pathways are enhanced and a carnosine synthetic pathway is introduced.

FIG. 8 illustrates results of measuring cell growth and productions of L-carnosine and glucose while culturing a recombinant strain (Car15) in which a pentose phosphate pathway and beta-alanine and histidine biosynthetic pathways are enhanced and a carnosine synthetic pathway is introduced through a fed-batch fermentation process.

### BEST MODE OF THE INVENTION

The present inventors developed a recombinant microorganism with improved productivities of carnosine, histidine, and beta-alanine by enhancing a pentose phosphate pathway, an L-histidine biosynthetic pathway, and a beta-alanine biosynthetic pathway and introducing a carnosine synthetic pathway through the redesign of metabolic pathways using metabolic engineering technology, and then completed the present invention.

Hereinafter, the present invention will be described in detail.

Accordingly, the present invention provides a recombinant microorganism for high production of carnosine, in which a pentose phosphate pathway is enhanced; and a mammal-derived carnosine synthase 1 (Carns1) gene is introduced.

In the present invention, the recombinant microorganism for high production of carnosine may be additionally enhanced with one or more of a histidine biosynthetic pathway and a beta-alanine biosynthetic pathway.

In the present invention, the carnosine synthase 1 (Carns1) gene is introduced into the recombinant microorganism to introduce the carnosine synthetic pathway, and the recombinant microorganism for high production of carnosine may continuously produce carnosine from beta-alanine and L-histidine biosynthesized within cells through the action of carnosine synthase produced by the expression of the introduced Carns1 gene.

In the present invention, the mammal-derived Carns1 gene may preferably be derived from a mouse (Mus musculus) and may consist of a nucleotide sequence represented by SEQ ID NO: 22. At this time, the gene may include a nucleotide sequence having 70% or more, preferably 80% or more, more preferably 90% or more, and most preferably 95%, 96%, 97%, 98%, and 99% or more of sequence homology with the nucleotide sequence represented by SEQ ID NO: 22.

In one embodiment of the present invention, in the Corynebacterium recombinant strain in which the pentose phosphate pathway is enhanced and the Carns1 gene is introduced, changes in production of carnosine were compared depending on whether the pentose phosphate pathway was enhanced. As a result, compared to a control strain (Car1) in which the pathway was not enhanced, in a strain (Car2) in which a promoter of an operon gene was replaced with a highly expressing synthetic promoter, the production of carnosine increased about two times. Additionally, it was confirmed that in a Car12 strain, in which the pentose phosphate pathway was further enhanced by changing an initiation codon of a pgi gene in the Car2 strain, the production of carnosine increased about 7 times compared to the control group (see Example 5).

In another embodiment of the present invention, in the Corynebacterium recombinant strain in which the pentose phosphate pathway was enhanced, after the beta-alanine and/or histidine biosynthetic pathway was further enhanced, the change in carnosine production was measured. As a result, it was confirmed that as compared with the strain in which only the pentose phosphate pathway was enhanced, the carnosine production increased about 7.2 times in a Car13 strain in which the beta-alanine biosynthetic pathway was further enhanced, about 2.4 times in a Car14 strain in which the histidine biosynthetic pathway was further enhanced, and about 10.5 times in a Car15 strain in which the beta-alanine and histidine biosynthetic pathways were enhanced together, which was the highest level (see Example 7).

Accordingly, the recombinant microorganism for high production of carnosine of the present invention may specifically include recombinant microorganisms in the range to be described below:
i) a recombinant microorganism in which the pentose phosphate pathway is enhanced and the Carns1 gene is introduced;
ii) a recombinant microorganism in which the pentose phosphate pathway and the beta-alanine biosynthetic pathway are enhanced and the Carns1 gene is introduced;
iii) a recombinant microorganism in which the pentose phosphate pathway and the histidine biosynthetic pathway are enhanced and the Carns1 gene is introduced; and
iv) a recombinant microorganism in which the pentose phosphate pathway and the beta-alanine and histidine biosynthetic pathways are enhanced and the Carns1 gene is introduced.

In the present invention, the recombinant microorganism for high production of carnosine may be most preferably a recombinant microorganism in which the pentose phosphate pathway and the beta-alanine and histidine biosynthetic pathways are enhanced and the Carns1 gene is introduced.

In addition, the present invention provides a recombinant microorganism for high production of histidine, in which one or more of pentose phosphate pathways and an L-histidine biosynthetic pathway are enhanced.

In one embodiment of the present invention, as a result of overexpressing the HisG and/or Rel gene using the recombinant vector in order to enhance the histidine biosynthetic pathway in Corynebacterium glutamicum, it could be seen that the production of histidine increased in a strain (Car7) overexpressing the genes simultaneously, and the histidine production increased at a high level in recombinant strains (Car8 to Car11) in which the pentose phosphate pathway was enhanced alone or in combination of the histidine biosynthetic pathway (see Example 6).

In addition, the present invention provides a recombinant microorganism for high production of beta-alanine, in which one or more of pentose phosphate pathways and a beta-alanine biosynthetic pathway are enhanced.

In one embodiment of the present invention, as a result of enhancing the beta-alanine biosynthetic pathway by further overexpressing a PanD gene using a recombinant vector in the Car12 strain in which the pentose phosphate pathway was enhanced and the Carns1 gene was introduced, it was confirmed that the production of beta-alanine increased about 4 times or higher in the Car13 strain (see Example 7).

Accordingly, in one embodiment of the present invention, the recombinant microorganism may be additionally introduced with a mammal-derived Carns1 gene.

In the present invention, the recombinant microorganism may preferably be derived from Corynebacterium glutamicum.

The pentose phosphate pathway (PPP) is also called a phosphogluconate pathway or a hexose monophosphate pathway, and a metabolic pathway of oxidizing glucose 6-phosphate (G-6-P) into pentose phosphate. The pentose phosphate pathway produces NADPH and a pentose derivative, ribose 5-phosphate (R-5-P), which is a precursor for the synthesis of nucleotides. Although the pentose phosphate pathway includes the oxidation of glucose, a main role of the pentose phosphate pathway is anabolism rather than catabolism.

In the present invention, the pentose phosphate pathway may be enhanced by the replacement existing promoters with a highly expressing synthetic promoter for overexpression of genes consisting of an operon, that is, transaldolase (tkt), transaldolase (tal), glucose-6-phosphate 1-dehydrogenase (zwf), glucose-6-phosphate 1-dehydrogenase (opcA), and 6-phosphogluconolactonase (pgl), ii) the change of a glucose-6-phosphate isomerase (pgi) gene to an initial codon, or a combination thereof.

In the present invention, the highly expressing synthetic promoter may be H36 consisting of a nucleotide sequence represented by SEQ ID NO: 3, but is not limited thereto as long as the promoter is a synthetic promoter capable of increasing the expression levels of the genes.

In the present invention, the initiation codon of the pgi gene may be preferably changed from ATG to GTG.

In the present invention, the histidine biosynthetic pathway may be enhanced by the overexpression of an ATP phosphoribosyltransferase (HisG) gene, the overexpression of a GTP pyrophosphokinase (Rel) gene, or a combination thereof.

In the present invention, the beta-alanine biosynthetic pathway may be enhanced by the overexpression of an aspartate 1-decarboxylase (PanD) gene.

The HisG, Rel, and PanD genes may consist of nucleotide sequences represented by SEQ ID NOs: 13, 14, and 19, respectively. At this time, the genes may include nucleotide sequences having 70% or more, preferably 80% or more, more preferably 90% or more, and most preferably 95%, 96%, 97%, 98%, and 99% or more of sequence homology with the nucleotide sequences represented by SEQ ID NOs: 13, 14, and 19.

Another aspect of the present invention provides a method for producing the recombinant microorganism for high production of carnosine including the following steps:
(a) enhancing pentose phosphate pathways through the replacement of a promoter of an operon-type gene with a highly expressing synthetic promoter, the change of a glucose-6-phosphate isomerase (pgi) gene into an initiation codon, or a combination thereof in microorganisms having glutamic acid productivity; and
(b) introducing a mammal-derived carnosine synthase 1 (Carns 1) gene.

In the present invention, the production method may further include the overexpression of the HisG (ATP phosphoribosyltransferase) and Rel (GTP pyrophosphokinase) genes, the overexpression of the PanD (Aspartate 1-decarboxylase) gene, or a combination thereof.

In addition, the present invention provides a method for producing a recombinant microorganism for high production of L-Histidine including enhancing pentose phosphate pathways through the replacement of a promoter of an operon-type gene with a highly expressing synthetic promoter, the change of a glucose-6-phosphate isomerase (pgi) gene into an initiation codon, or a combination thereof in microorganisms having glutamic acid productivity.

In addition, the method for producing the recombinant microorganism for high production of L-Histidine includes enhancing pentose phosphate pathways through the replacement of a promoter of an operon-type gene with a highly expressing synthetic promoter, the change of a glucose-6-phosphate isomerase (pgi) gene into an initiation codon, or a combination thereof in microorganisms having glutamic acid productivity; and overexpressing one or more of HisG (ATP phosphoribosyltransferase) gene and Rel (GTP pyrophosphokinase) gene.

In addition, the present invention provides a method for producing a recombinant microorganism for high production of beta-alanine including enhancing pentose phosphate pathways through the replacement of a promoter of an operon-type gene with a highly expressing synthetic promoter, the change of a glucose-6-phosphate isomerase (pgi) gene into an initiation codon, or a combination thereof in microorganisms having glutamic acid productivity.

In addition, the method for producing the recombinant microorganism for high production of beta-alanine includes enhancing pentose phosphate pathways through the replacement of a promoter of an operon-type gene with a highly expressing synthetic promoter, the change of a glucose-6-phosphate isomerase (pgi) gene into an initiation codon, or a combination thereof in microorganisms having glutamic acid productivity; and overexpressing a PanD (Aspartate 1-decarboxylase) gene.

In one embodiment of the present invention, the method for producing the recombinant microorganism for high production of beta-alanine may further include introducing a mammal-derived Carns1 (Carnosine synthase 1) gene, preferably expressing both the Carns1 gene and the PanD gene in one recombinant vector.

The term “recombinant microorganism” used in the present invention refers to a microorganism in which a genetic material has been recombined by genetic recombinant DNA introduced through genetic recombination technology. The genetic recombinant DNA refers to DNA constructed by combining DNA (carrier) replicable within any cell and heterogeneous DNA in vitro using enzymes, etc.

In the present invention, preferably, the recombinant microorganism may be a microorganism having glutamic acid productivity, in which the productivity of carnosine, histidine, and beta-alanine is improved through genetic recombination technology, and the microorganism having glutamic acid productivity may be preferably Corynebacterium glutamicum.

As used in the present invention, the term “vector” means a DNA construct containing a DNA sequence operably linked to a suitable control sequence capable of expressing DNA in a suitable host. The vector may be a plasmid, a phage particle, or simply, a potential genomic insert. When transformed into the suitable host, the vector may replicate and function independently of a host genome, or may be integrated into a genome itself in some cases. Since the plasmid is the most commonly used form of the current vector, the “plasmid” and the “vector” are sometimes used interchangeably in the specification of the present invention.

For the purposes of the present invention, it is preferred to use plasmid vectors. A typical plasmid vector that may be used for the purpose has a structure including (a) a replication origin that allows efficient replication to contain several to hundreds of plasmid vectors per host cell, (b) an antibiotic resistance gene that allows selection of host cells transformed with a plasmid vector, and (c) a restriction enzyme cleavage site into which a foreign DNA fragment may be inserted. Even if a suitable restriction enzyme cleavage site does not exist, the vector and the foreign DNA may be easily ligated using a synthetic oligonucleotide adapter or linker according to a conventional method.

As used in the present invention, the term “recombinant vector” is a recombinant carrier into which a heterologous DNA fragment is inserted, and generally refers to a double-stranded DNA fragment. Here, the heterologous DNA refers to heteromorphous DNA, which is DNA that is not found naturally in a host cell. When the recombinant vector is once present in the host cell, the recombinant vector may replicate independently of host chromosomal DNA and several copies of the vector and its inserted (heterologous) DNA may be produced. Thereafter, the recombinant vector may be transformed or transfected into a host cell.

As used in the present invention, the term “transformation” means that DNA is introduced into a host so that the DNA is an extrachromosomal factor or replicable by chromosomal integration completion. Of course, it should be understood that all of the vectors do not exhibit functions equally in expressing the DNA sequences of the present invention. Likewise, all of the hosts do not exhibit functions equally for the same expression system. However, those skilled in the art may make appropriate selections among various vectors, expression control sequences, and hosts without departing from the scope of the present invention without undue experimental burden. For example, in selecting the vectors, the host needs to be considered, which is because the vectors need to replicate therein. The copy number of the vector, the ability to control the copy number, and the expression of other proteins encoded by the corresponding vector, such as antibiotic markers, need also to be considered.

The “transformation” or “transfection” may be used with various techniques commonly used to introduce exogenous nucleic acids (DNA or RNA) into prokaryotic or eukaryotic host cells, such as electroporation, calcium phosphate precipitation, DEAE-dextran transfection or lipofection, etc., but is not limited thereto and may be appropriately selected and used by those skilled in the art.

As well-known in the art, in order to increase the expression level of a transfected gene in the host cell, the corresponding gene needs to be operably linked to transcriptional and translational expression control sequences that exhibit the functions in the selected expression host. Preferably, the expression control sequence and the corresponding gene are included in one recombinant vector including both a bacterial selection marker and a replication origin. When the host cell is the eukaryotic cell, the recombinant vector needs to further contain an expression marker useful in the eukaryotic expression host.

In the present invention, the preferred host cell may be a prokaryotic cell, and preferably the prokaryotic host cell may include C. glutamicum ATCC 13826, C. glutamicum ATCC 13032, C. glutamicum 13059, C. glutamicum ATCC 14067, C. glutamicum ATCC. 13761, C. glutamicum ATCC 13058, C. glutamicum ATCC 13745, etc., but is not limited thereto. Also, E. coli strains such as E. coli DH5a, E. coli JM101, E. coli TOP10, E. coli K12, E. coli W3110, E. coli X1776, E. coli XL1-Blue (Stratagene), E. coli B, and E. coli BL21 and strains included in other prokaryotic species and genera may be used.

Yet another aspect of the present invention provides a method for producing carnosine including culturing the recombinant microorganism for high production of carnosine.

Further, the present invention provides a method for producing L-Histidine including culturing the recombinant microorganism for high production of histidine.

Further, the present invention provides a method for producing beta-alanine including culturing the recombinant microorganism for high production of beta-alanine.

In the present invention, conditions for culturing such as a temperature, an environment, a medium, a culture container, etc. are not specifically limited as long as the conditions allow for the growth of the recombinant microorganism according to the present invention, preferably recombinant Corynebacterium glutamicum, and the culturing may be performed for an appropriate time by setting and applying appropriate conditions according to the corresponding recombinant microorganism, a type of material to be produced and an amount to be obtained by those skilled in the art.

In one embodiment of the present invention, a recombinant strain (Car15) in which the pentose phosphate pathway, the beta-alanine synthetic pathway, and the histidine synthetic pathway were enhanced and the carnosine synthetic pathway was introduced was culture on an agar plate in a standing incubator, first and second pre-cultured in a shake flask, and then subjected to fed-batch fermentation for 48 hours. As a result, it was confirmed that the production of carnosine was further increased two times or higher compared to the case of only the flask culture with the same strain.

Accordingly, in the present invention, the culturing of the carnosine producing strain may preferably be performed through fed-batch fermentation, but is not limited thereto and may be appropriately selected and applied by those skilled in the art.

As used in the present invention, the term “fed-batch fermentation” is a type of liquid culture among methods of culturing microorganisms and is a fed-batch method classified according to a supply method of a substrate. Specifically, the substrate concentration in the culture medium may be arbitrarily controlled by a culture method for intermittently supplying the medium. Since the substrate is added at an appropriate rate and not leaked, the substrate may be freely controlled by maintaining a balance between the amount of substrate to be supplied and the amount consumed by the microorganisms. In the present invention, the fermentation process was performed at 30° C. and 200 to 400 rpm, and pH 6.8 was maintained until the end of culture, but specific conditions are not limited to the above range.

### MODES OF THE INVENTION

Hereinafter, preferred examples will be proposed in order to help in understanding of the present invention. However, the following Examples are just provided to more easily understand the present invention, and the contents of the present invention are not limited by the following Examples.

### EXAMPLES

### Example 1. Construction of Recombinant Strain with Enhanced Pentose Phosphate Pathway

1-1. Obtaining of Strain Replaced with Synthetic Promoter

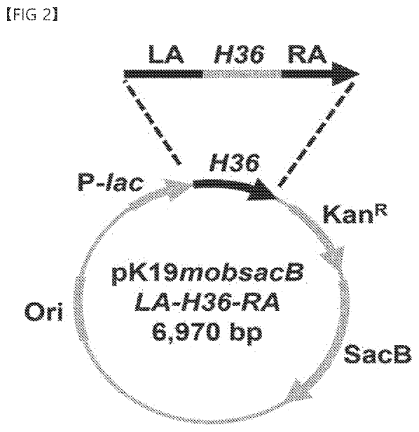

The present inventors attempted to replace a natural promoter with a H36 promoter as a highly expressing synthetic promoter for overexpression of related genes consisting of an operon, that is, transketolase (tkt), transaldolase (tal), glucose-6-phosphate 1-dehydrogenase (zwf), glucose-6-phosphate dehydrogenase assembly protein (OpcA), and 6-phosphogluconolactonase (pgl) in order to develop a Corynebacterium strain with an enhanced pentose phosphate pathway, and to this end, constructed an integration vector as shown in FIG. 2 according to the following process.

Specifically, in order to construct the vector, a left arm sequence of 587 bp (SEQ ID NO: 1) was obtained through PCR amplification using forward and reverse primers(tkt LA F (EcoR1) and tkt LA R (Xma1)) including a restriction enzyme sequence from the genomic DNA of Corynebacterium glutamicum ATCC 13032. In addition, a right arm of 600 bp (SEQ ID NO: 2) was additionally obtained from the left arm through PCR amplification using forward and reverse primers(tkt RA F(Xba1) and tkt RA R(Sal1)). Subsequently, in order to replace the natural promoter, a H36 promoter of 74 bp (SEQ ID NO: 3) was additionally obtained through PCR amplification using forward and reverse primers(H36 BamH1 F and H36 Xba1 R) including the restriction enzyme sequence. Thereafter, in order to construct a pK19mobsacB-H36 vector of FIG. 2, the obtained gene(left arm-H36 promoter-right arm) and the pK19mobsacB vector were each treated with restriction enzymes, a ligation reaction was performed, and the genes were introduced to an Escherichia coli (E. Coli) DH5a strain and a Corynebacterium glutamicum strain and transformed. The transformed Corynebacterium glutamicum strain was cultured overnight on a BHI plate containing kanamycin and then colony PCR was performed to confirm first recombination, and the strain in which the first recombination was confirmed was cultured under a condition of 30° C. on an LB plate containing 10% sucrose for performing second recombination. The colonies formed after culturing were inoculated and cultured on the LB plate containing kanamycin and the plate containing 10% sucrose, respectively. Thereafter, colonies growing only in the 10% sucrose medium were selected and the strain with the replaced promoter was obtained by confirming that the natural promoter was replaced with a highly expressing promoter.

1-2. Additional Obtaining of Strain with Changed Initiation Codon of Pgi Gene

In the Corynebacterium strain in which the pentose phosphate pathway was enhanced by replacing the promoter through Example 1-1, in order to further enhance the pathway, it was attempted to change the initiation codon of the pgi gene to gtg using a CRISPR system.

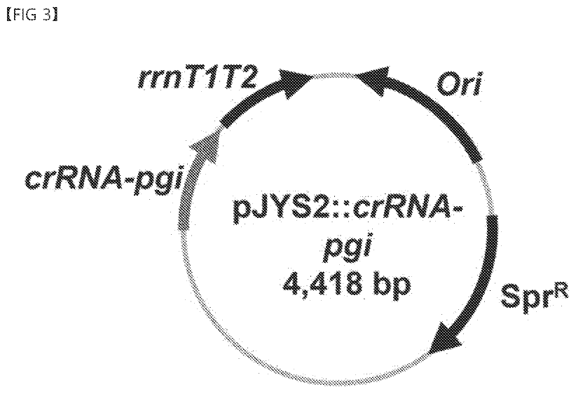

For this purpose, a pJYS2::crRNA-pgi vector illustrated in FIG. 3 was constructed according to the following method. Specifically, to construct the vector, PCR amplification was performed using forward and reverse primers(crRNA pgi F and crRNA pgi R) to obtain a crRNA pgi fragment of 75 bp (SEQ ID NO: 4). Thereafter, the pJYS2 vector was treated with a restriction enzyme and the ligation reaction of the crRNA pgi fragment was performed using a Gibson assembly method, and the constructed vector was introduced into the E. Coli DH5a strain to induce transformation. Next, for CRISPR, the Corynebacterium glutamicum strain was introduced with the pJYS1 vector containing Cas12a and transformed to produce competent cells, and a pJYS2_crRNA_pgi vector and a lagging strand were introduced. Thereafter, in order to confirm changes in the nucleotide sequence of the initiation codon within the pgi gene, colony PCR was performed using colonies formed in the BHISG medium containing kanamycin and spectinomycin, and then sequencing was performed, and after confirming the change in nucleotide sequence, the CRISPR vector was removed through curing to obtain a final strain with the enhanced pentose phosphate pathway. Primer sequences used in Examples 1-1 and 1-2 were summarized in Table 1 below.

TABLE 1

| Target | Direction | Sequence (5′-3′) | SEQ ID NO: |
|---|---|---|---|
| tkt LA | Forward | ATATGAATTCTTAAGTTGTGAGTCCTTAT | 5 |
| | Reverse | ATACCCGGGCAATCTTAAGTCTGGGA | 6 |
| tkt RA | Forward | ATATCTAGATTGACCACCTTGACGCTGTCACCTG | 7 |
| | Reverse | ATAGTCGACCAGCTGCTGGGTGCCAGCGA | 8 |
| H36 | Forward | ATAGGATCCTCTATCTGGTGCCCTAAACG | 9 |
| | Reverse | ATATCTAGACATGCTACTCCTACCAACCA | 10 |
| crRNA pgi | Forward | CTAGGTATAATGGATCGAATTTCTACTGTTGTAGATATGGCGGACATT | 11 |
| | Reverse | TTTATTTAAATGGATCTGGGTGGTCGAAATGTCCGCCATATCTACAAC | 12 |

### Example 2. Construction of Recombinant Strain with Enhanced Histidine Biosynthetic Pathway

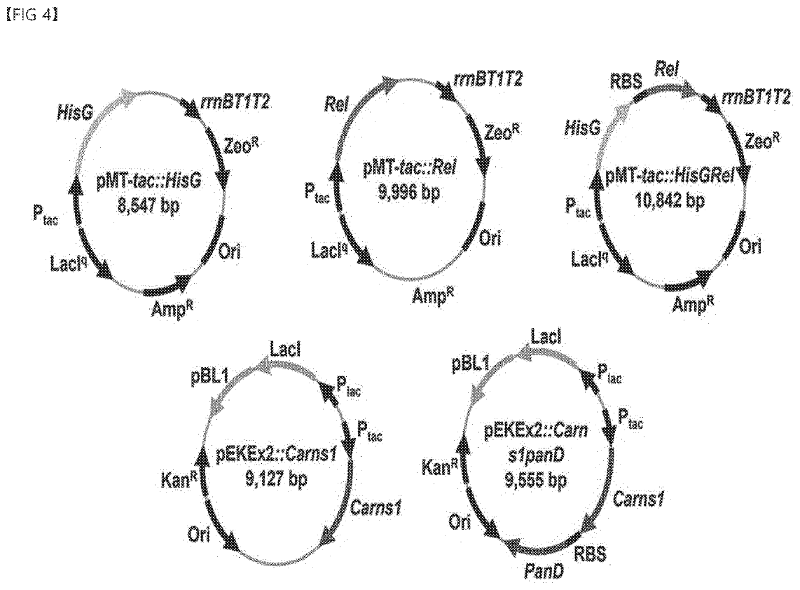

The present inventors used a method for introducing HisG and Rel genes to the Corynebacterium strain with the enhanced pentose phosphate pathway prepared in Example 1, in order to additionally enhance the histidine synthetic pathway. To this end, pMT_tac::HisG, pMT_tac::Rel, and pMT_tac::HisG-Rel, which were recombinant vectors for transformation of Corynebacterium glutamicum overexpressing HisG, Rel, HisG, and Rel shown in FIG. 4, were constructed according to the following process.

Specifically, for the construction of pMT_tac::HisG, PCR was performed using forward and reverse primers(HisG F (Cla1) and HisG R (BamH1)) containing the corresponding restriction enzyme sequence of the pMT_tac vector from the genomic DNA of Corynebacterium glutamicum, and as a result, a HisG gene of 846 bp (SEQ ID NO: 13) was obtained. In addition, for the construction of pMT_tac::Rel, PCR was performed using forward and reverse primers(Rel F(Sma1) and Rel R (Xma1)) containing the corresponding restriction enzyme sequence of the pMT_tac vector from the genomic DNA of Corynebacterium glutamicum, and as a result, a Rel gene of 2283 bp (SEQ ID NO: 14) was obtained. The forward primer for constructing the pMT_tac::Rel was inserted with a ribosome binding site (RBS) sequence (AAGGAGATATAG), which was a specific sequence complementarily binding to the ribosome required for translation of the gene to help the translation of the Rel gene after the translation of the HisG gene was completed.

Thereafter, in order to construct a recombinant vector expressing each gene, the genes amplified through PCR and the pMT-tac vector were treated with the restriction enzyme, subjected to the ligation reaction, and then introduced into the E. Coli DH5a strain and the Corynebacterium glutamicum strain and transformed. In addition, in order to introduce the Rel gene into the constructed pMT-tac::HisG vector, the pMT-tac::HisG vector was treated with the restriction enzyme, and then a ligation reaction was performed to produce a pMT_tac::HisGRel vector expressing both HisG and Rel. The constructed vector was introduced into the E. Coli DH5a strain and the Corynebacterium glutamicum strain and transformed, and the primer sequences used in the experiment were shown in Table 2 below.

TABLE 2

| Gene | Direction | Sequence (5′-3′) | SEQ ID NO: |
|---|---|---|---|
| HisG | Forward | ATAATCGATATGTTGAAAATCGCTGTCCC | 15 |
| | Reverse | ATAGGATCCCTAGATGCGGGCGATGC | 16 |
| Rel | Forward | ATACCCGGGAAGGAGATATAGATGAGTCTGGAGCGCAACAC | 17 |
| | Reverse | ATAGCGGCCGCCTAGCCACCCGAGGTCAC | 18 |

### Example 3. Construction of Recombinant Strain with Enhanced Pentose Phosphate Pathway and Beta-Alanine Biosynthetic Pathway

The present inventors constructed a vector for overexpressing a PanD gene according to the following process, in order to construct a strain in which a beta-alanine pathway was enhanced additionally in the Corynebacterium strain with the enhanced pentose phosphate pathway produced in Example 1.

Specifically, a PanD gene (SEQ ID NO: 19) was obtained through PCR amplification using forward and reverse primers (PanD F (EcoR1): 5′-GGTACCGAGCTCGAATTATGCTGCGCACCATCCT-3′ (SEQ ID NO: 20), and PanD R (EcoR1): 5′-AAAACGACGGCCAGTGGAATTCTAAATGCTTCTCGACGTCAAAAGC-3′ (SEQ ID NO: 21)) from the genomic DNA of Corynebacterium glutamicum. Next, in order to construct a recombinant vector overexpressing the PanD gene, a pEKEx2 vector was treated with a restriction enzyme, subjected to a ligation reaction through Gibson assembly, and then introduced into the E. Coli DH5a strain and the Corynebacterium glutamicum strain and transformed.

### Example 4. Construction of Recombinant Strain Introduced with Carnosine Biosynthetic Pathway

The present inventors attempted to introduce a mammal-derived Carns1 gene to introduce the carnosine biosynthetic pathway into the Corynebacterium glutamicum strain or the recombinant Corynebacterium strains constructed in Examples 1 to 3. For this purpose, the Carns1 gene was amplified through PCR using forward and reverse primers (Carns1 F(Sal1): 5′-ATAGTCGACATGTGCTTGGCAAAGCAGAAG-3′ (SEQ ID NO: 23), Carns1 R(EcoR1): 5′-ATAGAATTCCTATTTGAAATGAGACAGGAAATGGGCAAC-3′ (SEQ ID NO: 24)) from the pCMV-SPORT6::Carns1 vector purchased from the Korea Human Gene Bank (SEQ ID NO: 22). Next, in order to construct a recombinant vector overexpressing the Carns1 gene illustrated in FIG. 4, the pEKEx2 vector or pEKEx2::panD vector was treated with a restriction enzyme, subjected to a ligation reaction, and then transformed into the E. Coli DH5a strain and the Corynebacterium glutamicum strain to construct a recombinant strain introduced with the carnosine biosynthetic pathway.

### Example 5. Confirmation of Changes in Production of Histidine and Carnosine Through Enhancement of Pentose Phosphate Pathway

The present inventors attempted to confirm the production of histidine and carnosine in the strain with the enhanced pentose phosphate pathway constructed in Example 1, and introduced a carnosine production pathway according to the method of Example 4 for the production of carnosine. Thereafter, the recombinant strain with the enhanced pentose phosphate pathway and the transformed strain with the enhanced pentose phosphate pathway and the introduced carnosine production pathway were cultured for 48 hours under conditions of 30° C. and 200 rpm in a 250 ml shaking conical flask containing 50 ml of a CGAF medium.

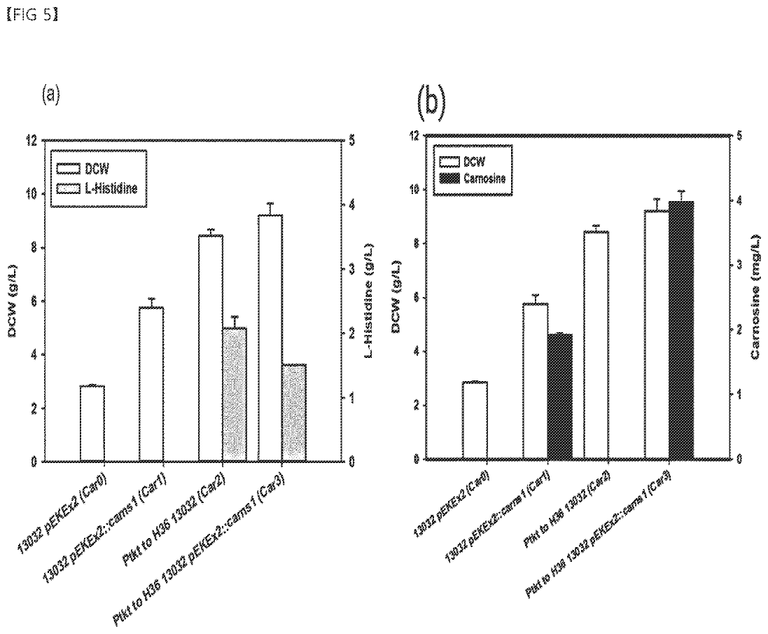

As a result, as illustrated in FIG. 5, first, when the production of L-histidine was measured, in a 13032 strain (Ptkt to H36 13032; Car2) in which the pentose phosphate pathway was enhanced, about 2.07 g/L of histidine was produced (summed inside and outside the cells), and in a control 13032 strain (13032 pEKEx2; Car0), 0.3 mg/L of histidine was produced (summed inside and outside the cell).

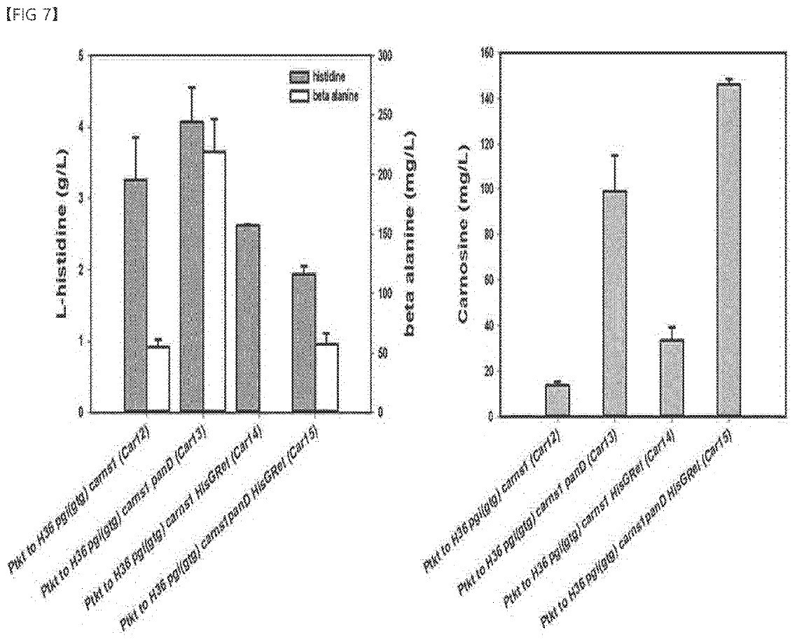

In addition, as a result of measuring the production of carnosine, it was confirmed that in a strain (13032 pEKEx2::Carns1; Car1) in which Carns1 was introduced in a control group (Car0) in which the pentose phosphate pathway was not enhanced, about 1.9 mg/L of carnosine was synthesized, and in a strain (Ptkt to H36 13032 pEKEx2::Carns1; Car3) in which Carns1 was introduced in the strain (Car2) in which the pentose phosphate pathway was enhanced, 3.9 mg/L of carnosine of about 2 times was synthesized. Additionally, in a recombinant strain (Ptkt to H36 pgi(gtg) Carns1; Car12) in which the pentose phosphate pathway was further enhanced and Carns1 was introduced through the change of the pgi initiation codon in addition to the change into the highly expressing synthetic promoter H36, as shown in FIG. 7, it was confirmed that the production of carnosine was increased about 7 times to 13.8 mg/L.

### Example 6. Confirmation of Increased Histidine Production Through Enhancement of Histidine Biosynthetic Pathway

The present inventors constructed a recombinant strain in which the histidine biosynthetic pathway was additionally enhanced by overexpressing HisG, Rel, or HisG and Rel according to the method of Example 2 in the strain with the enhanced pentose phosphate pathway obtained in Example 1, and then measured the production of histidine. All transformed strains were cultured for 48 hours under conditions of 30° C. and 200 rpm in a 250 ml shaking conical flask containing 50 ml of a CGAF medium.

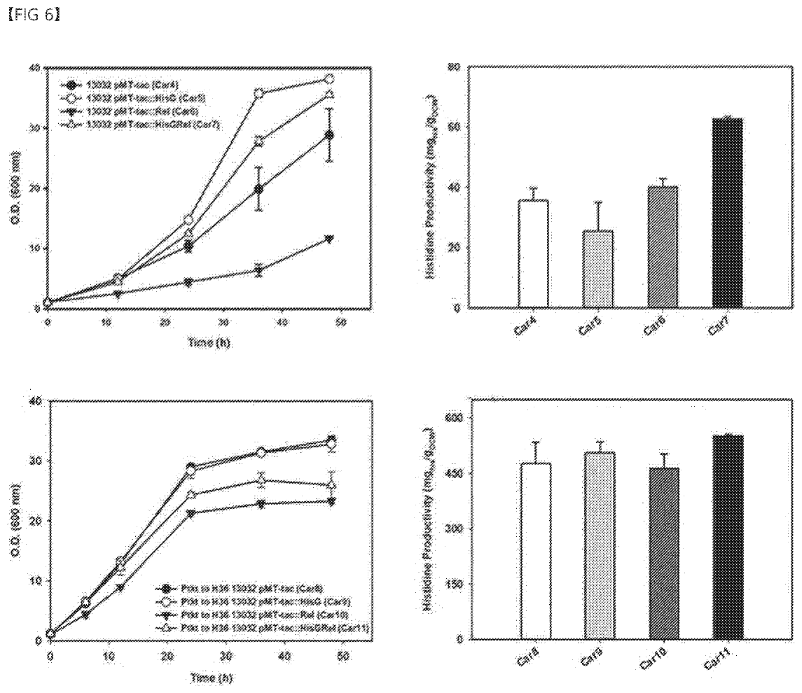

As a result, as shown in FIG. 6, it was confirmed that as compared with a control strain (13032 pMT-tac; Car4) in the recombinant strains Car4 to Car7 in which the pentose phosphate pathway was not enhanced, in a recombinant strain (13032 pMT-tac::HisGRel; Car7) in which the histidine biosynthetic pathway was enhanced through the overexpression of HisG and Rel, the production of histidine was increased. In addition, it was confirmed that in recombinant strains Car8 to Car11 in which the pentose phosphate pathway was enhanced, the production of histidine was significantly increased overall, and in a recombinant strain (Ptkt to H36 13032 pMT-tac::HisGRel; Car11) in which the pentose phosphate pathway was enhanced and HisG and Rel were overexpressed, the productivity of histidine was highest. From the result, it could be seen that the production of histidine was increased through the enhancement of the pentose phosphate pathway and the histidine biosynthetic pathway.

### Example 7. Confirmation of Increased Carnosine Production Through Enhancement of Beta-Alanine and/or Histidine Biosynthetic Pathway

The present inventors measured and compared the productions of histidine, beta-alanine, and carnosine in a recombinant strain (Ptkt to H36 pgi(gtg) Carns1; Car12) in which a pentose phosphate pathway was enhanced and a carnosine synthetic pathway was introduced by introducing Carns1; a recombinant strain (Ptkt to H36 pgi(gtg) Carns1 panD; Car13) in which a pentose phosphate pathway and a beta-alanine synthetic pathway were enhanced and a carnosine synthetic pathway was introduced; a recombinant strain (Ptkt to H36 pgi(gtg) Carns1 HisGRel; Car14) in which a pentose phosphate pathway and a histidine synthetic pathway were enhanced and a carnosine synthetic pathway was introduced; and a recombinant strain (Ptkt to H36 pgi(gtg) Carns1 panD HisGRel; Car15) in which a pentose phosphate pathway and beta-alanine and histidine synthetic pathways were enhanced and a carnosine synthetic pathway was introduced, respectively. The transformants were cultured for 48 hours under conditions of 30° C. and 200 rpm in a 250 ml shaking conical flask containing 50 ml of a CGAF medium.

As a result, as illustrated in FIG. 7, it was shown that as compared with the control Car12 in which only the pentose phosphate pathway was enhanced and the carnosine synthetic pathway was introduced, in a recombinant strain Car13, in which a beta-alanine production-related panD gene was additionally overexpressed, beta-alanine increased 4 times to 219.17 mg/L, and carnosine increased 7.2 times to about 99 mg/L. Meanwhile, it was confirmed that in the recombinant strain (Car14) in which the pentose phosphate pathway and the histidine synthetic pathway were enhanced, compared to the control Car12 strain, the production of histidine decreased, but the production of carnosine increased about 2.4 times to about 33 mg/L. Finally, in the recombinant strain (Car15) in which the pentose phosphate pathway, the beta-alanine synthetic pathway, and the histidine synthetic pathway were enhanced, ultimately, 145 mg/L of the highest production of carnosine was measured.

From the result, it could be seen that it was possible to increase the production of carnosine to the highest level by enhancing the pentose phosphate pathway, the histidine synthetic pathway and the beta-alanine synthetic pathway and introducing the carnosine synthetic pathway.

### Example 8. Confirmation of Increased Carnosine Production Through Fed-Batch Culture

The present inventors found a method for further increasing the carnosine production in the recombinant strain (Car15) in which the pentose phosphate pathway, the histidine synthetic pathway and the beta-alanine synthetic pathway were enhanced and the carnosine synthetic pathway was introduced, which was confirmed to produce the carnosine in the highest level in Example 7.

To this end, based on previous research results of the present inventors, the strain was cultured by fed-batch culture to increase the production of carnosine. Specifically, before the fed-batch culture, the Car15 strain was cultured in a standing incubator for 24 hours under a condition of 30° C. on a BHISG agar plate, and then first pre-cultured for 24 hours under conditions of 30° C. and 200 rpm in a 100 ml shaking conical flask containing 20 ml of a preculture medium (10 g yeast extract, 10 g peptone, 2 g urea, 2.5 g NaCl, 40 g glucose and 0.1 mg biotine per liter). Next, for the second preculture, the cultured strain was inoculated into a 250 ml shaking conical flask containing 50 ml of the preculture medium of the same composition as above, and then cultured for 24 hours under conditions of 30° C. and 200 rpm. Thereafter, for fed-batch culture, the cultured strain was cultured in a fermenter containing a 2 L CGAF medium under conditions of 30° C. and 200 to 400 rpm for 48 hours, and the pH was maintained at 6.8.

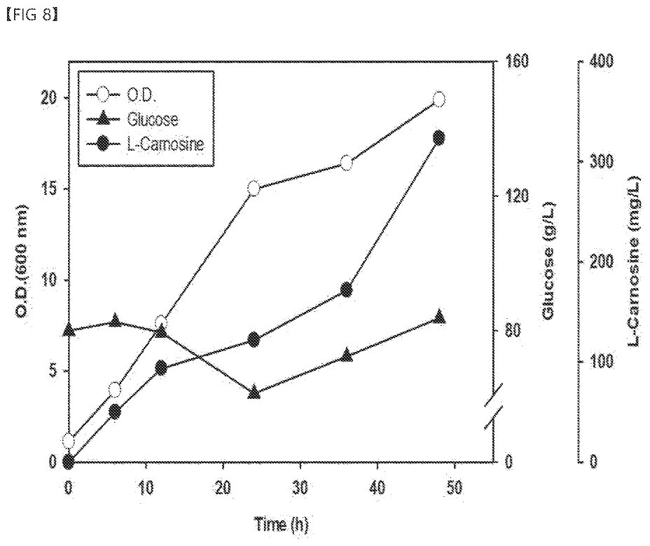

Thereafter, as a result of measuring the production of carnosine in the fed-batch cultured Car15 strain, as may be seen in FIG. 8, it was confirmed that the production of carnosine significantly increased, and then ultimately, 323.26 mg/L of the highest level of carnosine was produced.

The aforementioned description of the present invention is used for exemplification, and it can be understood by those skilled in the art that the present invention can be easily modified in other detailed forms without changing the technical spirit or requisite features of the present invention. Therefore, it should be appreciated that the embodiments described above are illustrative in all aspects and are not restricted.

1-31. (canceled)

32. A recombinant microorganism for high production of carnosine, wherein a pentose phosphate pathway is enhanced; and
a mammal-derived carnosine synthase 1 (Carns1) gene is introduced.

33. The recombinant microorganism of claim 32, wherein the recombinant microorganism is additionally enhanced with one or more of an L-histidine biosynthetic pathway and a beta-alanine biosynthetic pathway.

34. The recombinant microorganism of claim 32, wherein the pentose phosphate pathway is enhanced by the replacement of a promoter of an operon-type gene with a highly expressing synthetic promoter, the replacement of an initiation codon of a glucose-6-phosphate isomerase (pgi) gene, or a combination thereof.

35. The recombinant microorganism of claim 34, wherein the highly expressing synthetic promoter is H36 and the initiation codon of the pgi gene is changed from ATG to GTG.

36. The recombinant microorganism of claim 32, wherein the Carns1 gene consists of a nucleotide sequence represented by SEQ ID NO: 22.

37. The recombinant microorganism of claim 33, wherein the histidine biosynthetic pathway is enhanced by the overexpression of a ATP phosphoribosyltransferase (HisG) gene, the overexpression of a GTP pyrophosphokinase (Rel) gene, or a combination thereof.

38. The recombinant microorganism of claim 37, wherein the HisG and Rel genes consist of nucleotide sequences represented by SEQ ID NOs: 13 and 14, respectively.

39. The recombinant microorganism of claim 33, wherein the beta-alanine biosynthetic pathway is enhanced by the overexpression of an aspartate 1-decarboxylase (PanD) gene.

40. The recombinant microorganism of claim 39, wherein the PanD gene consists of a nucleotide sequence represented by SEQ ID NO: 19.

41. The recombinant microorganism of claim 32, wherein the recombinant microorganism is derived from Corynebacterium glutamicum.

42. A recombinant microorganism for high production of histidine, wherein one or more of pentose phosphate pathways and an L-histidine biosynthetic pathway are enhanced.

43. A recombinant microorganism for high production of beta-alanine, wherein one or more of pentose phosphate pathways and a beta-alanine biosynthetic pathway are enhanced.

44. The recombinant microorganism of claim 42, wherein the pentose phosphate pathway is enhanced by the replacement of a promoter of an operon-type gene with a highly expressing synthetic promoter, the replacement of an initiation codon of a glucose-6-phosphate isomerase (pgi) gene, or a combination thereof.

45. The recombinant microorganism of claim 44, wherein the highly expressing synthetic promoter is H36.

46. The recombinant microorganism of claim 44, wherein the initiation codon of the pgi gene is changed from ATG to GTG.

47. The recombinant microorganism of claim 42, wherein the histidine biosynthetic pathway is enhanced by the overexpression of a ATP phosphoribosyltransferase (HisG) gene, the overexpression of a GTP pyrophosphokinase (Rel) gene, or a combination thereof.

48. The recombinant microorganism of claim 47, wherein the HisG and Rel genes consist of nucleotide sequences represented by SEQ ID NOs: 13 and 14, respectively.

49. The recombinant microorganism of claim 43, wherein the beta-alanine biosynthetic pathway is enhanced by the overexpression of an aspartate 1-decarboxylase (PanD) gene.

50. The recombinant microorganism of claim 49, wherein the PanD gene consists of a nucleotide sequence represented by SEQ ID NO: 19.

51. The recombinant microorganism of claim 42, wherein the recombinant microorganism is derived from Corynebacterium glutamicum.


SEQUENCE LISTING (24 sequences)

SEQ ID NO: 1 - DNA, 587 bp, Artificial Sequence
  tkt left arm
ttaagttgtgagtccttattatttaaatatccctgcggtgagtgtgcaccttgcgttgaaggcccagactctgacagaagcgtcagagtgtttactcaagacattttctaagacacacggcaaattagtcggatgaagttaattaaaagttcccgaatcaatctttttaatgttttcaaaccatttgaaggtgtgctgacccaggtggacgccaacctttaaaaagcttcagacttttatttccacttcataaaaactgcctgtgacgattccgttaaagattgtgccaaatcactgcgcaaaactcgcgcggaaccagaccttgccatgctatcgcctattcacactatttgagtaatcggaaatagatgggtgtagacgcttgattggcggacggttcacagcggacgatttcaggccctcgtagctcgagagtttgaaggggtccgattcgttccgttcgtgacgctttgtgaggttttttgacgttgcaccgtattgcttgccgaacatttttcttttcctttcggtttttcgagaattttcacctacaaaagcccacgtcacagctcccagacttaagattg

SEQ ID NO: 2 - DNA, 600 bp, Artificial Sequence
  tkt right arm
ttgaccaccttgacgctgtcacctgaacttcaggcgctcactgtacgcaattacccctctgattggtccgatgtggacaccaaggctgtagacactgttcgtgtcctcgctgcagacgctgtagaaaactgtggctccggccacccaggcaccgcaatgagcctggctccccttgcatacaccttgtaccagcgggttatgaacgtagatccacaggacaccaactgggcaggccgtgaccgcttcgttctttcttgtggccactcctctttgacccagtacatccagctttacttgggtggattcggccttgagatggatgacctgaaggctctgcgcacctgggattccttgaccccaggacaccctgagtaccgccacaccaagggcgttgagatcaccactggccctcttggccagggtcttgcatctgcagttggtatggccatggctgctcgtcgtgagcgtggcctattcgacccaaccgctgctgagggcgaatccccattcgaccaccacatctacgtcattgcttctgatggtgacctgcaggaaggtgtcacctctgaggcatcctccatcgctggcacccagcagctg

SEQ ID NO: 3 - DNA, 74 bp, Artificial Sequence
  H36 promoter
tctatctggtgccctaaacgggggaatattaacgggcccagggtggtcgcaccttggttggtaggagtagcatg

SEQ ID NO: 4 - DNA, 75 bp, Artificial Sequence
  crRNA pgi
ctaggtataatggatcgaatttctactgttgtagatatggcggacatttcgaccacccagatccatttaaataaa

SEQ ID NO: 5 - DNA, 29 bp, Artificial Sequence
  tkt LA_Forward
atatgaattcttaagttgtgagtccttat

SEQ ID NO: 6 - DNA, 26 bp, Artificial Sequence
  tkt LA_Reverse
atacccgggcaatcttaagtctggga

SEQ ID NO: 7 - DNA, 34 bp, Artificial Sequence
  tkt RA_Forward
atatctagattgaccaccttgacgctgtcacctg

SEQ ID NO: 8 - DNA, 29 bp, Artificial Sequence
  tkt RA_Reverse
atagtcgaccagctgctgggtgccagcga

SEQ ID NO: 9 - DNA, 29 bp, Artificial Sequence
  H36_Forward
ataggatcctctatctggtgccctaaacg

SEQ ID NO: 10 - DNA, 29 bp, Artificial Sequence
  H36_Reverse
atatctagacatgctactcctaccaacca

SEQ ID NO: 11 - DNA, 48 bp, Artificial Sequence
  crRNA pgi_Forward
ctaggtataatggatcgaatttctactgttgtagatatggcggacatt

SEQ ID NO: 12 - DNA, 48 bp, Artificial Sequence
  crRNA pgi_Reverse
tttatttaaatggatctgggtggtcgaaatgtccgccatatctacaac

SEQ ID NO: 13 - DNA, 846 bp, Corynebacterium glutamicum_HisG
atgttgaaaatcgctgtcccaaacaaaggctcgctgtccgagcgcgccatggaaatcctcgccgaagcaggctacgcaggccgtggagattccaaatccctcaacgtttttgatgaagcaaacaacgttgaattcttcttccttcgccctaaagatatcgccatctacgttgctggtggccagctcgatttgggtatcaccggccgcgaccttgctcgcgattcccaggctgatgtccacgaagttctttccctcggcttcggttcctccactttccgttacgcagcaccagctgatgaagagtggagcatcgaaaagctcgacggcaagcgcatcgctacctcttaccccaaccttgttcgcgatgacctcgcagcacgtgggctttccgctgaggtgctccgcctcgacggtgcagtagaggtatccatcaagcttggtgtcgcagatgccatcgccgatgttgtatccaccggccgcacgctgcgtcagcaaggtcttgcacctttcggcgaggttctgtgcacctctgaggctgtcattgttggccgcaaggatgaaaaggtcaccccagagcagcagatcctgcttcgccgcatccagggaattttgcacgcgcagaacttcctcatgctggattacaacgtcgaccgcgacaacctggacgctgccactgcagtaaccccaggcttatccggcccaacggtatccccactggcacgcgacaactgggttgctgtacgcgccatggtgccacgcaggtcagctaacgccatcatggataagcttgctggactcggcgctgaagccatcctggcttctgaaatccgcatcgcccgcatctag

SEQ ID NO: 14 - DNA, 2283 bp, Corynebacterium glutamicum_Rel
atgagtctggagcgcaacacacaaaaatcttccatgggtgtgcgaagcatgtcagccaggcttgcccgcagcctcacaggaaaccgcgttcgcaccaaccctgtgctggatccgctgctgagcatccaccggcaatttcacccacgcgccgacgtacaagtgttggaacgtgcatatgacaccgcggaacgtcttcatgatggtgtgattcgaaaatcgggcgatccgtatattacccacccgttggctgtcgccaccatcgccgcggaaatcggcatggacaccaccacgctcgtcgcagccttgttgcatgacacggtggaagacaccgactactctttggacgatctcacccgagatttcggagaagaagttgccaggcttgtcgacggtgtcaccaagctcgacaaagtcgcactaggtgctgccgcggaggccgaaacgattcgcaaaatgatcgtcgccatgagccaggacccccgcgtgctggtgattaaagtggccgaccgtttgcacaatatgcgcaccatgcggttcctgccgccggaaaagcaagctaaaaaagcacgccaaacccttgaagtgattgctcctttggcacaccgcctgggcatggccagcgtgaaatgggaattggaagatctatcctttgccattttgtaccccaagaagtacgaagagatcgtgcgtcttgttgccgaccgcgcgccctctagagaccggtacctcaaagaaattattgatcaagtcaccggtggcttgcgcgaaaacaacatcgcggcagaagtgcttggtcgaccaaagcactactggtctatctatcaaaagatgatcgttcgcggtcgtgattttgacgatatttttgatcttgttggcatccgcatcctggtagacaacgtgaacaactgttacgccgccatcggtgtcgtgcactccctgttcaatgctctgcctggccgattcaaagactatatttcagccccgcgcttcggtgtctaccaatccctgcacaccaccgtgatgggacctggcggtaagcctctggaagttcaggcacgtacccacgacatgcactacaacgccgaattcggcattgcagcgcactggcgatacaaagaaaccaaaggcagccacagtggcgagcaagccgaagtggatcaaatggcgtggatgcgccaacttctggactggcaaaaagaagcagccgaccccaacgagttcctggacagcctgcgctacgatctgacttccaagcagatcttcgtgttcacacccaaaggtgatgtggtcaacctgccggtgaactccaccccggtggacttcgcctacgcggtgcacaccgaagtggggcaccgctgcatcggcgccaaaatcaacggcaaactggtcgctttggaaacgaaactcaaatccggcgatcgtgttgaagtctttacctccaaggaccaaaacgctggcccaagtaggggatggcaagaatttgttgtctcacctcgtgcaaaggccaagattcgccagtggtttgccaaggaacgacgcgaagaatacctagaagccggacgcgatgcgctggcagcagttattcagcgtggcggcctgccaatgcaccgcttgttcaccgcgtcctccatgaagacggtggcaacagagctgcactacccagatgtagatgcgctctacacagccatcggctccggttctgtatctgcgcaacacgtagtcaaccgtctcatggctatctttggtgacgaagaagatgccgaagacgcattggttgcacgcaccccattcagcgagctggtcaactcccgtgccaccacggaaagcagcaccggcatcctggtcgaaggcagcccagatgtcatggctaagctcgctaaatgctgtatgccagtgccaggagatgaaatctttggattcgtcacccgtggtggcggtgtctccgtacaccgaacagactgcacgaatgtggaaaagctcaaagaagagccagaacgcattgtctccgtctcctgggcttcggaaggtcaaggttcagtattctctgccacactgcagcttgaagcacttgatcgcgcaggcctgctctttgagctcacccgcgtaatcaacgaacaaaaggtctccgttaccgcaatgaactcccattgctcagaagaccgcgtagccaccgtgcgcttcacctttgcggtctctgacaccaagcagttgggatccctgatgacacagctgcgcaatgccgaaggagtgtttgatgtctaccgagtgacctcgggtggctag

SEQ ID NO: 15 - DNA, 29 bp, Artificial Sequence
  HisG_Forward
ataatcgatatgttgaaaatcgctgtccc

SEQ ID NO: 16 - DNA, 26 bp, Artificial Sequence
  HisG_Reverse
ataggatccctagatgcgggcgatgc

SEQ ID NO: 17 - DNA, 41 bp, Artificial Sequence
  Rel_Forward
atacccgggaaggagatatagatgagtctggagcgcaacac

SEQ ID NO: 18 - DNA, 29 bp, Artificial Sequence
  Rel_Reverse
atagcggccgcctagccacccgaggtcac

SEQ ID NO: 19 - DNA, 411 bp, Corynebacterium glutamicum_PanD
atgctgcgcaccatcctcggaagtaagattcaccgagccactgtcactcaagctgatctagattatgttggctctgtaaccatcgacgccgacctggttcacgccgccggattgatcgaaggcgaaaaagttgccatcgtagacatcaccaacggcgctcgtctggaaacttatgtcattgtgggcgacgccggaacgggcaatatttgcatcaatggtgccgctgcacaccttattaatcctggcgatcttgtgatcatcatgagctaccttcaggcaactgatgcggaagccaaggcgtatgagccaaagattgtgcacgtggacgccgacaaccgcatcgttgcgctcggcaacgatcttgcggaagcactacctggatccgggcttttgacgtcgagaagcatttag

SEQ ID NO: 20 - DNA, 34 bp, Artificial Sequence
  PanD_Forward
ggtaccgagctcgaattatgctgcgcaccatcct

SEQ ID NO: 21 - DNA, 46 bp, Artificial Sequence
  PanD_Reverse
aaaacgacggccagtggaattctaaatgcttctcgacgtcaaaagc

SEQ ID NO: 22 - DNA, 993 bp, Mus musculus_Carns1
atgtgcttggcaaagcagaagagccgcacacagctacatctgttgcggtgccagggcccaccctggccttcgacctctctccatgctgtggcctgttgcccattggagaatgaggctgatgtggagagagccatttaccaggtacccctgccaggtgtgatgaaactggaatttgggtcaggagcagtaggcgtgcagctggtgaaggatgggcctcagtgccgtgagcacttttcccggatcctccatgacctgcagggtgaggctgaccaccctggcatcgggctgggctggggcaatgctatgctgctgatggagtttgttgagggcacagagcatgacgtggacctggtggtgttcggtggacggttgctggctgctttcgtctccgataatggccccacaaggctgcctggcttcacagagactgcagcctgcatgcccactgggctagcaccagagcaggaggcccagatggtgcaggcagcctttcgctgctgcctgggctgtgggctgctggatggggttttcaatgtggagctcaagatgactggggctgggccaaggctcatcgagattaaccctcgaatgggaggcttctaccttcgagactggatcctagagctctatggtgtggatttgcttctggcttctacaatggtggcctgtggcctgcagcctgccttgcctgctcaccctcgtgcccgtggctacctagtgggcatcatgtgcctggtgtcccagcacctacagctgctgagttccccgagcagtagagagactctgcaaaccctccatgaccaggggcaattacgactcaacctgctggaggaggccctgatacctggccagtatgaggagccctactgcaatgtggcctgtgctgggcccagccctgctgaggcctgtcaccgcctgctaggcatctgtcagggcttgggcattgacaggcccaactaccctgttgcccatttcctgtctcatttcaaatag

SEQ ID NO: 23 - DNA, 30 bp, Artificial Sequence
  Carns1_Forward
atagtcgacatgtgcttggcaaagcagaag

SEQ ID NO: 24 - DNA, 39 bp, Artificial Sequence
  Carns1_Reverse
atagaattcctatttgaaatgagacaggaaatgggcaac